(19) United States
(12) Patent Application Publication
(10) Pub. No.: US 2006/0258857 A1
(43) Pub. Date: Nov. 16, 2006

(54) Process for making steroidal compounds

(76) Inventors: Reinerus G. Gieling, Nijmegen (NL); Marinus Bernard Groen, Oss (NL)

Correspondence Address: MARK R. BUSCHER;SYNTHON IP INC, 7130 HERITAGE VILLAGE PLAZA, STE 202, GAINESVILLE, VA, 20155, US

(21) Appl. No.: 11/377,396

(22) Filed: Mar. 17, 2006

Related U.S. Application Data
(60) Provisional application No. 60/662,548, filed on Mar. 17, 2005.

(51) Int. Cl. C07J 21/00

(52) U.S. Cl. 540/44

(57) ABSTRACT

The reaction of a nitrile of formula (2) with water in the presence of a noble metal catalyst, such as a platinum complex of dialkyl- or diaryl-phosphine oxides, to form an amide of formula (3) is useful in the synthesis of steroidal compounds such as eplerenone: 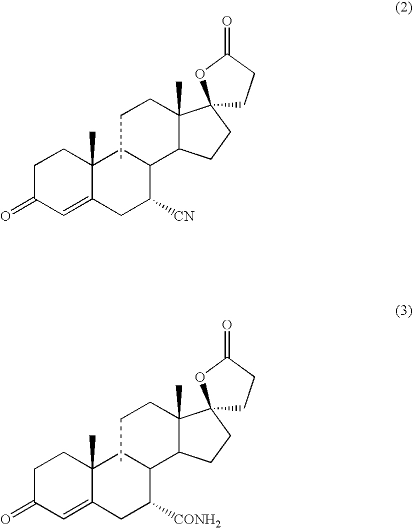 wherein the dotted line represents a single bond, a double bond, or an epoxy-bond.

This application claims the benefit of priority under 35 U.S.C. § 119(e) from U.S. provisional application Ser. No. 60/662,548, filed Mar. 17, 2005, the entire contents of which are incorporated herein by reference.

### BACKGROUND OF THE INVENTION

The present invention relates to the formation of various steroidal compounds and to certain novel compounds of the process.

Steroid-type compounds, herein referred to as “steroidal,” have found many uses in pharmaceuticals. A basic steroidal backbone structure, shown below as structure A, and its well known sub-genus, shown below as structure B, are conventionally called 20-spiroxan and canrenone, respectively.

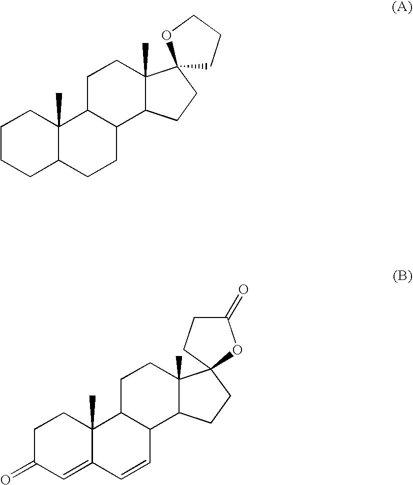

Derivatives of these compounds can have advantageous pharmaceutical effects. One such derivative that has good pharmaceutical utility is eplerenone (9α, 11α-epoxy-7α-methoxycarbonyl-20-spirox-4-ene-3,21-dione), also called epoxymexrenone, which has the formula (1). 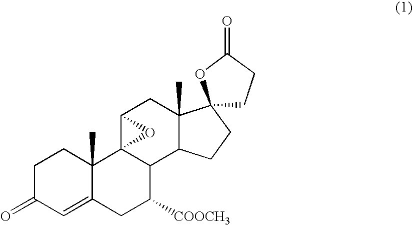
Eplerenone is an orally-active aldosterone antagonist that is used for the treatment of hypertension and congestive heart failure (CHF).

Eplerenone has been disclosed in EP 122232. A convenient process for making eplerenone disclosed therein is shown in the Scheme 1, which is represented below, where the starting compound is 20-spiroxa-4,6,9(11)-triene-3,21-dione (herein also referred to as “Δ^{9,11}-canrenone”) (cf. J. Med. Chem., 6, 732-735 (1963)).

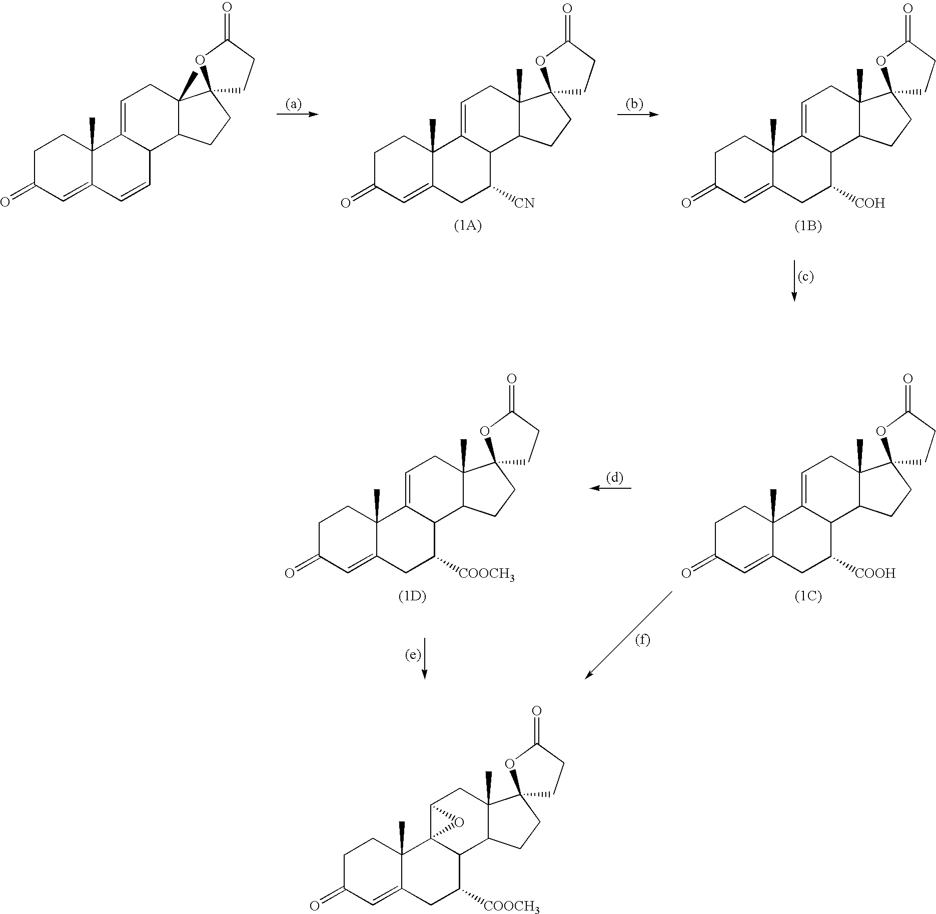

To the respective steps:

(a) Diethylaluminium cyanide solution is added to a THF solution of the starting Δ^{9,11}-canrenone. After elaboration and chromatographic treatment, 7α-cyano-20-spiroxa-4,9(11)-diene-3,21-dione(1A) was obtained.

(b) The above cyano derivative reacted with 20% (w/v) solution of diisobutylaluminium hydride in toluene (10° C., 30 min). After elaboration and extraction with chloroform; 7α-formyl-20-spiroxa-4,9(11)-diene-3,21-dione (1B) is obtained from the organic phase.

(c) The above crude formyl compound in acetone is oxidized with 8N solution of CrO₃ in aqueous H₂SO₄. After elaboration and extraction with chloroform, 3,21-dioxo-20-spiroxa-4,9(11)diene-7α-carboxylic acid (1C) is obtained.

(d) An ethereal diazomethane solution was added to a methylene chloride solution of the above acid. After elaboration and crystallization from methylene chloride/ether/petroleum ether, 7α-methoxycarbonyl-20-spiroxa-4,9(11)-diene-3,21-dione (ID) is obtained.

(e) Epoxidation of the above 9(11)-unsaturated spiroxene derivative by m-chloroperbenzoic acid provides eplerenone.

(f) An alternative epoxidation method enables the of esterification of the acid and the epoxidation to be carried out in a single step based on the reaction of the acid (1C) with m-chloroperbenzoic acid followed with addition of etheric diazomethane solution.

Alternative conditions of the above epoxidation procedure were shown in WO 97/21720 and WO 98/25948.

According to WO 2003/082895, eplerenone can also be prepared from the Δ^{9,11}-canrenone intermediate by an alternative six-step synthesis described in “Chart O” of the patent document (Scheme 2, steps B1-B6). The scheme is represented below.

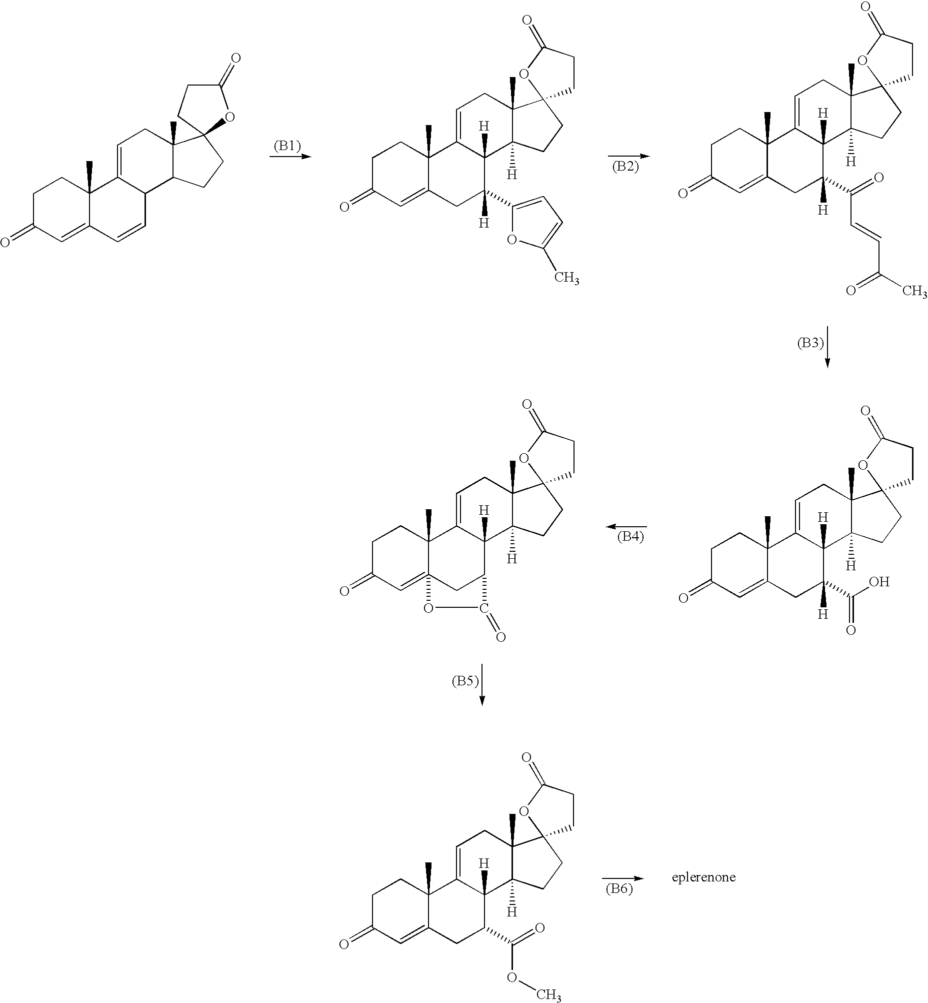

Eplerenone was prepared from Δ^{9,11}-canrenone via conjugate addition of 2-methylfuran in the presence of CH₃NO₂ and of BF₃·OEt₂, (step B1) with subsequent furane ring cleavage with dibromantin acetate in aqueous HCl (step B2). The synthesis was continued by ozonolysis with dimethylsulfide, resulting to a 7-carboxylic acid intermediate (step B3); with subsequent reaction with p-toluenesulfonic acid (step B4), then treated with bicarbonate and dimethyl sulfate (step B5), resulting to already known enester intermediate (see compound 1D in the Scheme 1 above). In the last step (B6), the enester underwent epoxidation reaction (using trichloroacetamide and hydrogen peroxide, in presence of ethanol and methyl ethyl ketone), giving eplerenone.

Any synthetic process comprising an introduction of a methyloxycarbonyl group into the position 7 of the spiroxane skeleton is faced with many problems arising from the sensitivity of the steroid skeleton. One problem is the ease at which epimerisation takes place at the position 7 in an alkaline environment. By said epimerisation, an undesired diastereomer may be formed. Furthermore, the lactone ring easily opens during various reaction conditions. Although the open ring may be closed back to the lactone ring under certain conditions, this step decreases the reaction yields, sometimes significantly. Furthermore, the double bond at position 4,5 is sensitive to a rearrangement into the position 5,6.

In summary, conventional processes useful for introducing the methoxycarbonyl group on a cyclohexane ring may be used only with care or modified dramatically in the presence of sensitive dienone and lactone systems.

In particular, when looking on the Scheme 1, it is remarkable that the steroid nitrile (1A) was not converted into the acid (1C) by the process of acidic or alkaline hydrolysis, which would appear to be a simple process. Instead, the steroid nitrile was converted into the carboxylic acid by a complicated reductive hydrolysis, followed by oxidation. This may suggest a problem with acidic or alkaline hydrolysis as a reaction step for converting the nitrile to the acid; else why to complicate matters.

Indeed, U.S. Pat. No. 3,890,304 teaches that the conversion of a steroidal nitrile, the compound (C), to the acid (E) may be achieved via corresponding amide (D) under conventional conditions of a step-wise hydrolysis at alkaline environment. 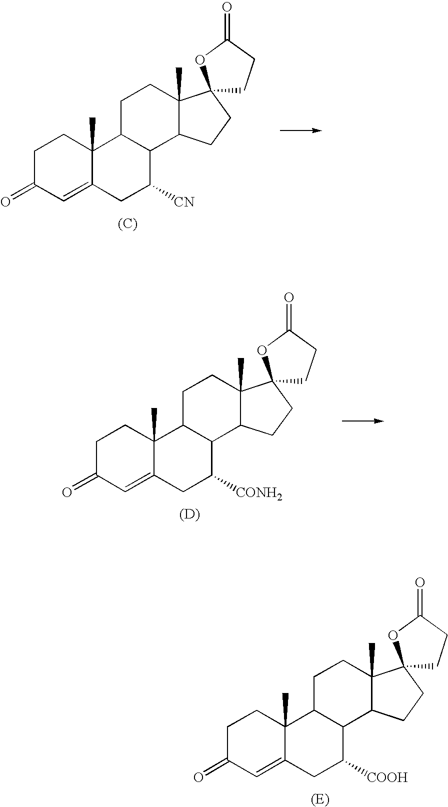
However no yields are reported and subsequent attempts to reproduce this process, in the course of making the present invention, yielded a mixture of products, from which the desired acid was not isolatable.

Moreover, the above process does not selectively hydrolyze to the amide, when it would be desirable to isolate and purify such amide.

Furthermore, this process has not been described for the compounds having the double bond in position 9,11 (such as delta-9,11-canrenone) or an epoxy-ring therein. Therefore, it appears that the conventional hydrolysis reaction is not available due to the sensitivity of the steroidal skeleton.

Another conventional hydrolytical process, an acidic hydrolysis of nitrites, has also been found to be problematic. Specifically, it leads to the undesired opening of the lactone ring and a subsequent irreversible rearrangement.

Similarly, a hydrolysis by hydrogen peroxide led only to the opening of the lactone ring. Without wishing to be bound by any theory, the inventors suggest that the pH of the reaction mixture plays an important role and conditions of conventional hydrolysis, which is normally performed either in alkaline or acidic environment, are too extreme for this particularly sensitive steroidal system.

Thus, an improvement in synthetic process leading to eplerenone and more generally to other 20-spioxane type steroids is desirable.

### SUMMARY OF THE INVENTION

The present invention is based on the discovery that an efficient conversion of a steroidal nitrile to a steroidal amide can be obtained. Accordingly, a first aspect of the present invention relates to a process, which comprises reacting a nitrile of formula (2) 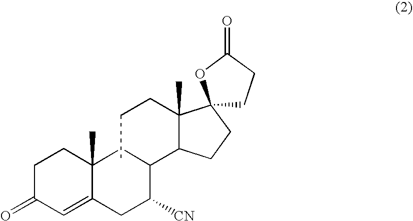
wherein the dotted line represents a single bond, a double bond, or an epoxy-bond, with water in the presence of a noble metal catalyst, especially a platinum complex of dialkyl- or diaryl-phosphine oxides to form an amide of formula (3) 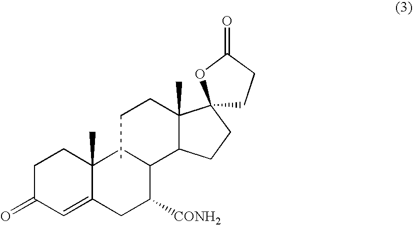
wherein the dotted line has the same meaning as in formula (2). Generally the catalyst is a platinum catalyst represented by the formula (4)
PtX(PR₂O)₂H(PR₂OH) (4)
wherein R represents a C₁-C₅ alkyl or phenyl group and X represents hydrogen or chlorine. Among the compounds of the general formula (4), the compounds (4A) and (4B) are preferred.

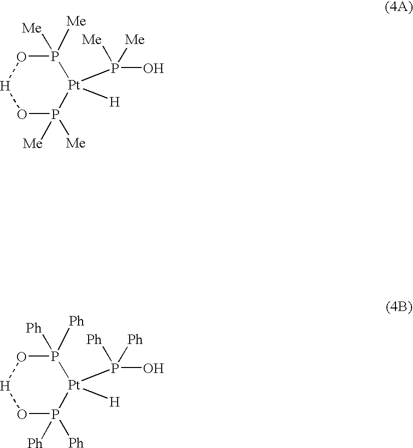

The process of the invention allows for a conversion of the nitrile group to an amide group without the disadvantages noted above. It can exhibit high yields and high selectivity, generally with no further conversion into an acid and with very low amounts of side products. Further, there is no need for the presence of additional acid or base in the reaction mixture, contrary to conventional hydrolysis. Thus, a pure amide of formula (3) may be readily isolated and characterized. The reaction of the invention can be used in making, inter alia, eplerenone; e.g. replacing the formation of the formyl derivative in the EP 122232 process with the formation of an amide derivative.

Another aspect of the invention relates to an improved process for producing eplerenone of formula (1) 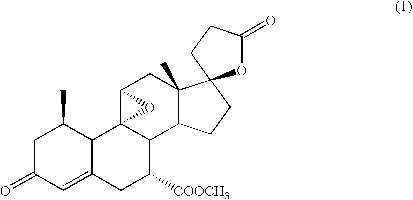
from a 20-spiroxan compound, the improvement of which comprises reacting a nitrile of formula (2) 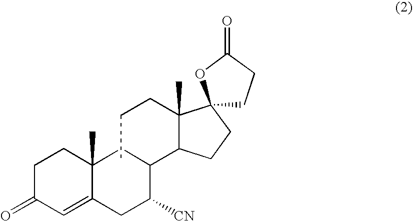
wherein the dotted line represents a single bond, a double bond, or an epoxy-bond, with water in the presence of a noble metal catalyst, especially a platinum complex of dialkyl- or diaryl-phosphine oxides, to form an amide of formula (3) 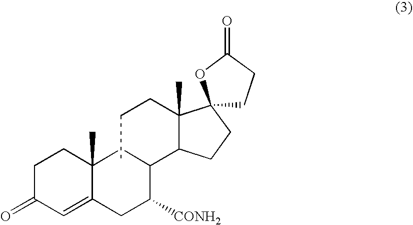
wherein the dotted line has the same meaning as in formula (2) and transforming said amide of formula (3) into eplerenone of formula (1).

A further aspect of the present invention relates to the novel compounds of formula (3) wherein the dotted line represents a double bond or an epoxy bond.

### DETAILED DESCRIPTION OF THE INVENTION

The invention provides a process of conversion of the nitrile of formula (2) into an amide of formula (3) using hydrolysis catalyzed by a water soluble noble metal homogeneous catalyst. In particular, the catalyst is a platinum (II) complex of dialkyl- or diaryl phospine oxides, which may be represented by the general formula (4).
PtX(PR₂O)₂H(PR₂OH) (4)
wherein R represents a C₁-C₅ alkyl, preferably methyl, or phenyl group and X represents hydrogen or chlorine.

The catalyst of formula (4) has been suggested for hydrolysis of nitrites generally in Ghaffar, Parkins, Tetrahedron Letters 36, 8657-8660 (1995). See also U.S. Pat. No. 5,932,756 and U.S. Pat. No. 6,133,478. Said documents also provide processes for making the catalysts. The preferred catalyst is represented by the formula (4A) or (4B), with (4A) being the more preferred.

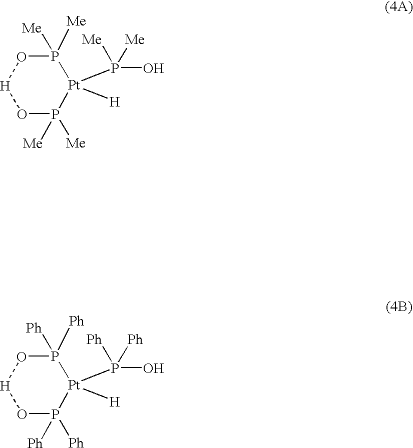

The starting nitrile of formula (2) may be provided by procedures known in the art and/or by techniques that are generally known in the art. For instance, the compound (2a) wherein the dotted line in (2) represents a single bond can be prepared according to U.S. Pat. No. 3,890,304. The compound (2b) wherein the dotted line in (2) represents a double bond can be prepared according to EP 122232 or Grob, J., Helv.Chim.Acta 80, 566-585 (1997). The compound (2c) wherein the dotted line in (2) represents an epoxy ring is a novel compound and may be prepared from the compound (2b) by an epoxidation process, for instance by treating the compound (2b) with hydrogen peroxide and potassium hydrogenphosphate in trichloroacetonitrile.

The conversion of the nitrile (2) into the amide (3) proceeds by contacting the compound (2), water, and the catalyst of formula (4) in a suitable solvent. The solvent may be water per se, but due to the limited solubility of the compound (2) in water, it is preferred that the reaction proceeds in a mixture of water with a water miscible organic solvent. Non-limiting examples of such water miscible solvents are C1-C4 aliphatic alcohols such as methanol or ethanol; C2-C6 aliphatic ketones such as acetone or methyl tert.butyl ketone; bipolar aprotic solvents such as dimethylformamide; cyclic ethers such as dioxane or tetrahydrofuran; etc. Logically, the solvent may not be a nitrile solvent.

The reaction may also proceed in a biphasic solvent system, i.e. in a mixture of water and a water immiscible (or partly miscible) organic solvent, preferably in the presence of a phase-transfer catalyst.

The reaction generally proceeds by dissolving or suspending the substrate in the liquid reaction medium comprising water within the solvent system, adding the catalyst and heating the mixture at an elevated temperature. Suitable amount of the catalyst is from 0.01 to 2.0 molar per cent based on the moles of the substrate (compound (2)). Suitable temperature is in the range of 50° C. up to reflux.

The time of reaction may be monitored by conventional analytical processes such as TLC or HPLC. Thereby, also the necessary reaction time may be determined. In practice, the reaction time is typically from 1 to 100 hours, but is not limited thereto.

After the reaction, the amide product is advantageously isolated from the reaction mixture. The amide product is insoluble or only sparingly soluble in water, thus it generally may precipitate from the aqueous environment. To the contrary, the catalyst is soluble in water so that it remains in the aqueous solution. Should a solvent mixture comprise an organic solvent, it is advantageous that the organic solvent is removed from the system and is replaced by water. In a simple arrangement, the solvent system is evaporated in vacuo and the residual solvent after evaporation is treated with water. The product precipitates as a solid, which may be isolated by filtration and optionally washed and dried. If necessary or desirable, the amide may be recrystallized, e.g. from water.

The amide compounds (3) are suitable intermediates for making various pharmaceutically useful products. The inventive process may be followed by a conversion of the resulting amidic product of formula (3) into an acid of formula (5), followed by esterification into an ester of formula (6), or by one step alcoholysis of the amide leading directly to the ester. 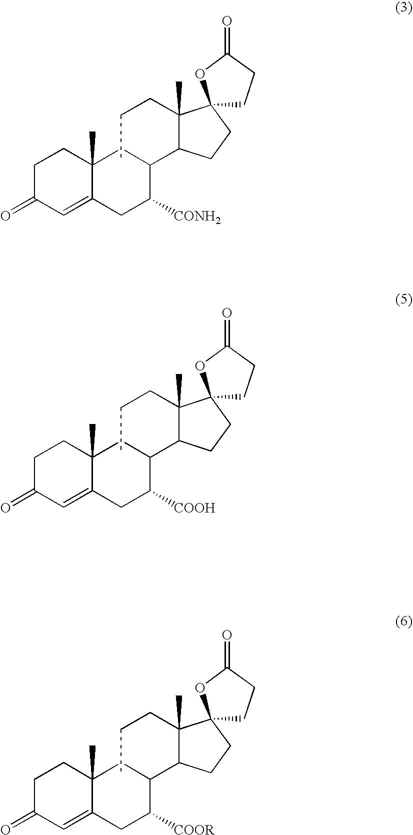
In formula (6), R represents a C1-C4 alkyl group.

Finally, if the product of formula (3), (5) or (6) does not contain the epoxy-ring, the ring may be formed by conventional methods in any stage during the conversion of the nitrile (2) to ester (6) or afterwards.

In particular, the inventive process may be used as part of an alternative and improved process for making eplerenone of formula (1). The use of these compounds and processes are herein after illustrated with respect to the process for making eplerenone of the formula (1); i.e. a species of the compound of formula (6). Several processes of converting the nitrile of the general formula (2), particularly compounds of formula (2b) and (2c), into eplerenone (1) via the amide (3) are shown in the following scheme:

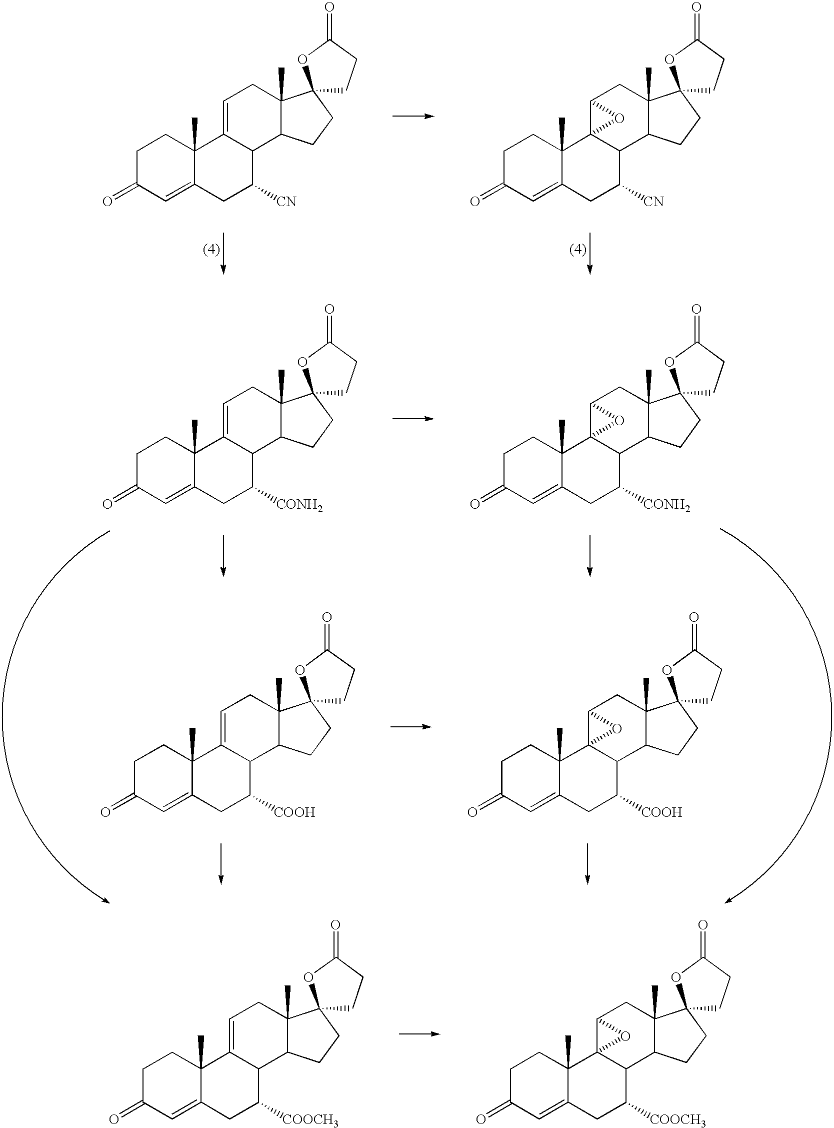

For example, once the amide (3) (either (3b) with a double bond or (3c) with an epoxy bond) is formed by the catalyzed hydrolysis of the correspond nitrite (2), it can be treated with nitrogen tetroxide or sodium nitrite at ambient temperature to give a nitrosamino compound, which can subsequently be converted to the acid of formula (5b) or (5c), respectively, by the action of potassium hydroxide at ambient temperature.

Esterification of the acid of formula (5) with a methylating reagent, such as diazomethane, dimethylsulfate or methyliodide, may give the methyl ester (6).

Alternatively, the amide (3) may also be hydrolyzed in an alkaline environment after protecting the 3-keto group by reduction with sodium borohydride or by ketalization with ethylene glycol or ethanedithiol. Deprotection after the hydrolysis, yielding the acid (5), can be accomplished by conventional methods and the methyl ester (6) is obtained by the esterification as described above.

In another approach, the amide (3) can be directly converted to the methyl ester (6) by the action of methoxide base, if necessary after activating the amide first with e.g. Di-tert-butyl dicarbonate or methanesulfonyl chloride, or by lactamization of the amide to the 5a-amino-γ-lactam compound.

The overall process of the present invention is particularly suitable for making eplerenone of formula (1), which is a subgenus of the compound of formula (6), wherein the dotted line represents an epoxy ring. Eplerenone may be prepared from the above amide compound (2), wherein the dotted line represents either a double bond or the epoxy-ring. Should the amide (2) comprise the epoxy ring, then the process of conversion into eplerenone is identical with that as disclosed above for conversion of (2) to (6) (i.e. the right hand side of the reaction scheme). If, however, the starting (2) comprises the double bond, a step of conversion of the double bond into the epoxy-ring must be added. Such conversion may proceed at any stage as shown in the scheme. Indeed, the epoxidation reaction itself or in combination with methyl esterification as a single step can be carried out by the methods and techniques disclosed in the prior art as described above (e.g., EP 122232 above, steps (f) or (e),).

The invention will be further described with reference to the following non-limiting examples.

### EXAMPLE 1

### Synthesis of 7α-Carbamoylcanrenone (3a)

162 mg 7α-cyanocanrenone (2a) and 4.5 mg of the Pt(H)(PMe₂OH)(PMe₂O)₂H catalyst (4A) were stirred in 0.6 ml ethanol/water (ratio 2/1) and heated on an oil bath of 80° C. Since not all nitrile dissolved, 1 ml ethanol was added. After a few minutes, a clear solution was obtained. After four hours of stirring (oil bath 80° C.), conversion was >95% according to HPLC. Ethanol was evaporated and the resulting slurry was freeze-dried, giving a yellow solid.

IR, ¹H-, ¹³C, ¹⁵N-NMR spectra confirmed the formation of the amide (3a).

### EXAMPLE 2

### Synthesis of 7α-Carbamoyl-9(11)^{Δ}-Canrenone (3b)

1.0 g of 7α-cyano-9(11)^{Δ}-canrenone (2b) was stirred in 20 ml of ethanol under nitrogen and heated to 60° C., giving a clear solution. The solution remained clear after addition of 2 ml water. 23 mg of the Pt(H)(PMe₂OH)(PMe₂O)₂H catalyst (4A) and 3 ml water were added. The reaction mixture was stirred at 67-73° C. for 21 hours, after which 98% of the starting material was converted according to HPLC. Ethanol was evaporated at reduced pressure and 4 ml water was added, giving an oil, which immediately solidified. The solid was filtered off and washed with 2 ml of water. The product was obtained as a sand-colored solid and dried at 40° C. under vacuum. ¹H- and ¹³C-NMR spectra were in accordance with the structure of amide (3b). Yield: 0.95 g (91%);

### EXAMPLE 3

### Synthesis of 7α-Carbamoyl-9(11)^{Δ}-Canrenone (3b)

1.96 g 7α-cyano-9(11)^{Δ}-canrenone (2b) was dissolved in 30 ml of ethanol under nitrogen by heating to 60° C. The solution remained clear after addition of 2 ml of water. 22.7 mg of the Pt(H)(PMe₂OH)(PMe₂O)₂H catalyst (4A) and 3 ml of water were added. The reaction mixture was stirred at 70° C. for 16 hours, after which 98% of the starting material was converted according to HPLC. The reaction mixture was allowed to cool to room temperature, followed by evaporation of approx. 25 ml ethanol at reduced pressure. 10 ml of water was added, resulting in formation of a small amount of crystals. The mixture was concentrated to a few ml smaller volume. An off-white solid crystallized. 10 ml of water was added and the solid was filtered off, washed with portions of 10 and 5 ml water, and then dried at 40° C. under vacuum. The amide was obtained as a sand-colored solid in a yield of 1.62 g (79%)

Infrared spectrum, ¹H- ¹³C and ¹⁵N -NMR spectra were in accordance with the structure of amide (3b)

An analytical sample was obtained by crystallization from the mother liquor (purity: >95% by area on HPLC, 254 nm); melting range: 253-257° C.

### EXAMPLE 4

### Synthesis of 7α-Carbamoyl-9(11)^{Δ}-canrenone (3b)

258 mg 7α-cyano-9(11)^{Δ}-canrenone (2b) was dissolved in 5 ml ethanol under nitrogen by heating to 60° C. The solution remained clear after addition of 1 ml water. 5 mg of the catalyst (Pt(H)(PPh₂OH)(PPh₂O)₂H) (4B) was added and the reaction mixture was stirred at 60° C. for 72 h. According to HPLC, reaction was complete. 10 ml of water was added to the cooled reaction mixture and after a while, white to off-white needles crystallized, which were filtered off and dried in vacuo at 40° C. for 20 h. A second and third crop crystallized from the mother liquor. Total yield: 222 mg. The crystals were identified by HPLC as the title compound (3b).

Each of the patents, articles, and publications mentioned above is incorporated herein by reference in its entirety. The invention having been thus described, it will be obvious to the worker skilled in the art that the same may be varied in many ways without departing from the spirit of the invention and all such modifications are included within the scope of the present invention as set forth in the following claims.

I claim:

1. A process, which comprises reacting a nitrile of formula (2) 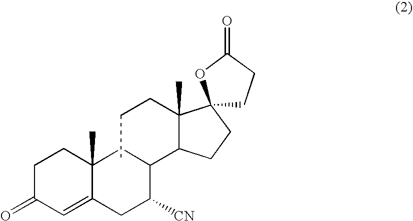
wherein the dotted line represents a single bond, a double bond, or an epoxy-bond, with water in the presence of a noble metal catalyst to form an amide of formula (3) 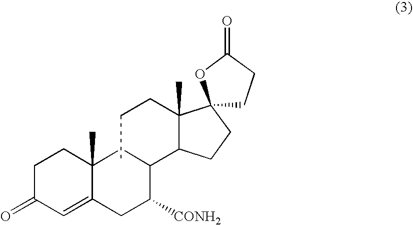
wherein the dotted line has the same meaning as in formula (2).

2. The process according to claim 1, wherein said reaction takes place in a solvent in which said nitrile of formula (2) is at least partially soluble.

3. The process according to claim 2, wherein said solvent comprises water.

4. The process according to claim 3, wherein said solvent is the source of said water used in the reaction.

5. The process according to claim 3, wherein said solvent comprises a mixture of water and alcohol, preferably methanol.

6. The process according to claim 1, wherein said catalyst is a platinum catalyst represented by the formula (4)
PtX(PR₂O)₂H(PR₂OH) (4)
wherein R represents a C₁-C₅ alkyl or phenyl group and X represents hydrogen or chlorine.

7. The process according to claim 6, wherein R is methyl or phenyl.

8. The process according to claim 7, wherein said catalyst is a compound of formula (4A) or (4B) 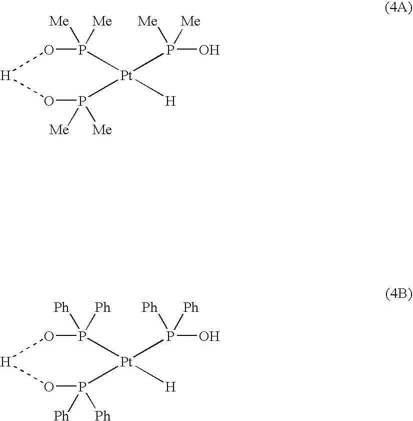
wherein Me represents a methyl group and Ph represents a phenyl group.

9. The process according to claim 1, wherein the dotted line in said nitrile of formula (2) is a double bond.

10. The process according to claim 1, wherein the dotted line in said nitrile of formula (2) is an epoxy bond.

11. The process according to claim 1, which further comprises converting said amide of formula (3) into an acid of formula (5) 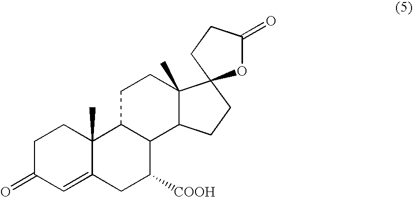
wherein the dotted line represents a single bond, a double bond, or an epoxy-bond.

12. The process according to claim 11, which further comprises esterifying said compound of formula (5) to form a compound of formula (6) 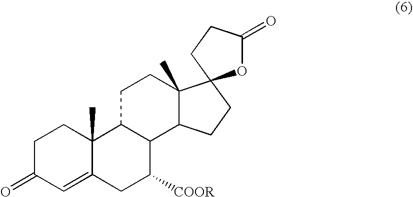
wherein the dotted line represents a single bond, a double bond, or an epoxy-bond and R represents a C₁-C₄ alkyl group.

13. The process according to claim 1, which further comprises directly converting said amide of formula (3) into an ester of formula (6) 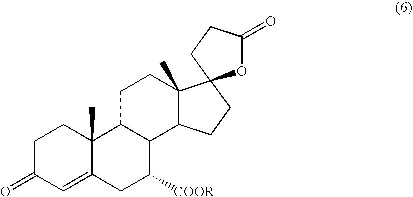
wherein the dotted line represents a single bond, a double bond, or an epoxy-bond and R represents a C₁-C₄ alkyl group.

14. The process according to claim 13, wherein R is a methyl group and the dotted line is a double bond and which further comprises converting said double bond to an epoxy bond to form eplerenone.

15. The process according to claim 13, wherein R is a methyl group and the dotted line represents an epoxy bond.

16. In a process for producing eplerenone of formula (1) 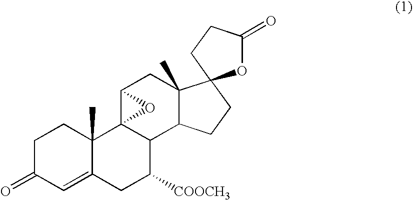
from a 20-spiroxan compound, the improvement of which comprises reacting a nitrile of formula (2) 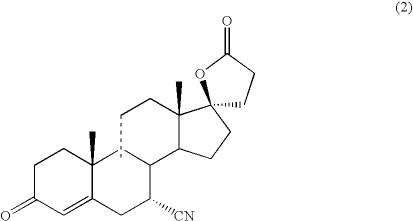
wherein the dotted line represents a single bond, a double bond, or an epoxy-bond, with water in the presence of a noble metal catalyst to form an amide of formula (3) 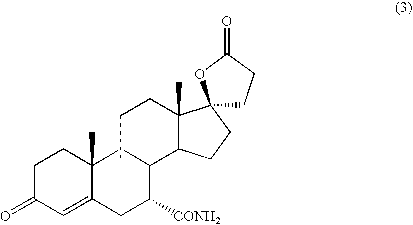
wherein the dotted line has the same meaning as in formula (2) and transforming said amide of formula (3) into eplerenone of formula (1).

17. The process according to claim 16, wherein said noble metal catalyst is a platinum complex of dialkyl- or diaryl-phosphine oxides.

18. The process according to claim 17, wherein said catalyst is a compound of formula (4A) or (4B) 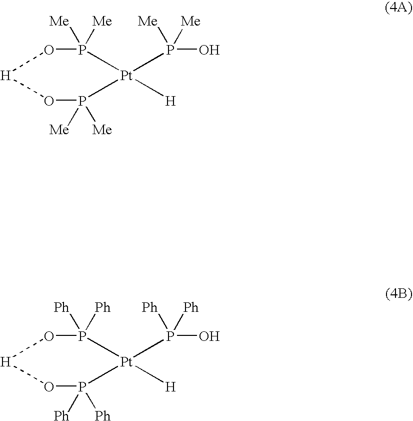
wherein Me represents a methyl group and Ph represents a phenyl group.

19. A compound of formula (3) 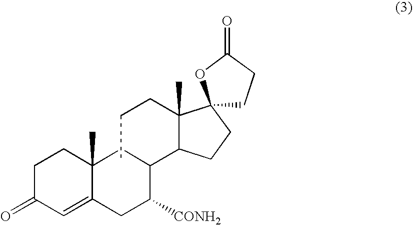
wherein the dotted line represents a double bond or an epoxy bond.
